(19) United States
(12) Patent Application Publication
(10) Pub. No.: US 2009/0312597 A1
(43) Pub. Date: Dec. 17, 2009

(54) Methods and devices for limiting gastric expansion

(76) Inventors: Eli Bar, Moshav Megadim (IL); Nissim Darvish, Hof HaCarmel (IL)

Correspondence Address: MARTIN D. MOYNIHAN d/b/a PRTSI, INC., P.O. BOX 16446, ARLINGTON, VA, 22215, US

(21) Appl. No.: 11/918,644

(22) PCT Filed: Apr. 11, 2006
(86) PCT No.: PCT/IL2006/000468

§ 371 Date: Oct. 17, 2007

(51) Int. Cl. A61F 2/00; A61B 17/12

(52) U.S. Cl. 600/37; 606/157

(57) ABSTRACT

Disclosed are methods and devices for limiting expansion in at least one portion of gastric tissue.

### FIELD OF THE INVENTION

The present invention relates generally to the field of gastric surgery. More specifically, the present invention provides methods and devices useful for deployment in and around gastric tissue, for example for the treatment of conditions related to the stomach.

### BACKGROUND OF THE INVENTION

The number of people being overweight or obese has reached epidemic proportions in the United States. In the last 10 years, obesity rates have increased by more than 60 percent among adults. In 1999, 61 percent of the adult population was either overweight or obese. The obesity epidemic impacts other diseases as well. For example, the incidence of type 2 diabetes, a major consequence of obesity, is on the rise. Among U.S. adults, diagnosed diabetes increased 49 percent from 1990 to 2000.

The rate of increase of young overweight people has been steeper. This is particularly troubling since many of the behaviors that lead to adult obesity are established during childhood. Just 10 years ago, type 2 diabetes was virtually unknown in children and adolescents. Indeed, the medical community commonly referred to the condition as “adult onset diabetes”.

More than 80 percent of people with type 2 diabetes are overweight. It is not known exactly why people who are overweight are more likely to suffer from type 2 diabetes. It may be that body cells in overweight individuals are less effective at processing sugar absorbed from the blood. This then increases stress on cells that produce insulin, a hormone that facilitates absorption, eventually resulting in failure of insulin producing cells.

Today, type 2 diabetes accounts for almost 50 percent of new cases of pediatric diabetes in some communities. Medical complications associated with obesity in children, in addition to type 2 diabetes, include sleep apnea, and asthma. Since 1980, the percentage of overweight children has nearly doubled, and the percentage of overweight adolescents has nearly tripled. Almost 9 million young Americans, or about 15 percent of all children, are overweight.

The incidence of many diseases increases with obesity, for example:

### Heart Disease

People who are overweight are more likely to suffer from a variety conditions that increase the risk of heart disease and stroke, including:

i) high blood pressure;

ii) high levels of triglycerides (blood fats);

iii) high levels of LDL cholesterol (a low density fat-like substance often called the “bad cholesterol”); and

iv) low levels of HDL cholesterol (the “good cholesterol”).

Overweight people, as a result of increased body fat, produce high levels of blood vessel inflammatory substances; believed to increase the risk of heart disease.

### Cancer

Overweight individuals appear to have a higher incidence of several types of cancer, including cancers of the colon, esophagus, and kidney. Overweight women appear to have a higher incident of uterine and postmenopausal breast cancer.

The greater incidence of cancer and cancer related mortalities in overweight individuals are not fully understood. One theory postulates that fat cells produce hormones that adversely affect cell growth, leading to cancer. It also appears that, high food consumption and/or physical inactivity, both associated with overweight individuals, may of them selves contribute to cancer risk.

### Sleep Apnea

The risk for sleep apnea is higher for people who are overweight. A person who is overweight may have more fat stored neck fat, making the airway smaller. A smaller airway can make breathing difficult, loud (snoring), or cease. In addition, neck fat may be responsible for fat-induced inflammation, causing localized symptoms that result in sleep apnea.

### Osteoarthritis

Extra weight may place extra pressure on joints and cartilage, causing joint wear. In addition, fat-induced inflammation may contribute to osteoarthritis.

### Gallbladder Disease

People who are overweight have a higher risk for developing gallbladder disease, including enlarged gallbladder and gallstones; the enlarged gallbladder being susceptible to malfunction and increased cholesterol, associated with high body weight, increasing the incidence of gallstones.

### Fatty Liver Disease

People who have diabetes or “pre-diabetes” (when blood sugar levels are higher than normal but not yet in the diabetic range) are more likely to have fatty liver disease than people without diabetes or pre-diabetes. It is not known why some people who are overweight and/or diabetic get fatty liver while others do not.

### Lowering Health Risk Through Weight Reduction

For someone overweight, losing as little as 5 percent of your body weight may lower your risk for several diseases, including heart disease and diabetes. For example, a person weighting 200 pounds will reduce heart disease risk by losing 10 pounds. Slow and steady weight loss of ½ to 2 pounds per week, and not more than 3 pounds per week, is the safest way to lose weight.

### Short Term Weight Loss Maintenance

Maintaining weight lost, even for the short term, requires changes in eating and physical activity habits, including:

i) choosing healthy foods, including vegetables, fruits, whole grains, low-fat meat and dairy products;

ii) eating just enough food to satisfy you;

iii) performing at least 30 minutes of moderate-intensity physical activity-like walking, on most days of the week, preferably every day.

Losing weight, as opposed to maintaining weight loss, may require more than 30 minutes of moderate physical activity daily.

### Long Term Weight Loss Maintenance

To lose weight and keep it off over time, it is not a simple task. People are desperately trying different weight loss solutions in order to lower health risks. Weight loss solutions, themselves, are often highly undesirable, requiring long-term draconian changes in life style.

Methods for achieving weight loss with minimal suffering are often centered on surgical operations designed to increase feelings of satiation. The most risk free surgical solutions are usually administered with minimal invasive technique, often through an endoscope.

In endoscopic weight loss solutions, the stomach or portions of gastric tissue are often restricted in their maximal expansion, thereby achieving satiation on small food quantities. Generally, endoscopic surgical tools are inserted through the oral cavity, pharynx, esophagus to the stomach, rather than through an abdominal incision, for example.

The most desirable endoscopic solutions dispense with cutting tissue, using gastric restriction devices instead. Gastric restriction devices that install without tissue cutting reduce surgical risk, recovery time, and procedural costs.

### PRIOR ART

A variety of stomach restriction devices are known.

Gastric anchors are known. For example Imran teaches a variety of anchor designs and methods of configuration, including: U.S. patent application Ser. No. 11/992,382, published on 30 Jun. 2005 as US 2005/0143784 A1, which teaches devices and methods to anchor sensors to a gastric portion; and U.S. patent application Ser. No. 10/991,648 published on 20 Jun. 2005 as US 2005/0143760 A1A1, which teaches devices and methods to interconnect anchor to gastric tissue and limit gastric expansion.

Gastric pouches are known, for example Stack et al, in U.S. patent application Ser. No. 10/345,666 published on 22 Jul. 2004 as US 2004/0143342 A1 teach a pouch that impedes passage of food through the stomach.

Gastric cages and bypass sleeves are known, for example Levine et at, teach a variety of devices, including gastric cages, sleeves and implementation instruments that are designed to limit gastric expansion, including: U.S. patent application Ser. No. 10/726,011 published 21 Apr. 2005 as US 2005/0085923 A1; U.S. patent application Ser. No. 10/811,293 published 4 Nov. 2004 as US 2004/0220682 A1; U.S. patent application Ser. No. 11/001,794 published 14 Apr. 2005 as US 2005/0080395 A1; U.S. patent application Ser. No. 11/001,812 published 2 Dec. 2004 as US 2004/0243195 A1; U.S. patent application Ser. No. 10/999,846 published 14 April 2005 as US 2005/0080491 A1; and U.S. patent application Ser. No. 10/000,899 published 7 Apr. 2005 as US 2005/0075622 A1.

The contents of all of the above applications are hereby incorporated by reference as if fully set forth herein.

### SUMMARY OF THE INVENTION

Embodiments of the present invention successfully address at least some of the shortcomings of the prior art by providing methods and devices for limiting gastric expansion so as to affect gastric motility, volume, hunger sensation and/or nutrient absorption.

According to the teachings of the present invention, there is provided a method for limiting expansion in at least one portion of gastric tissue comprising, looping an elongate primary element around a substantially continuous first gastric tissue portion having a surface such that a leading portion of the primary element exits a first gastric tissue surface at a first point, a trailing portion of the primary element exits a second gastric tissue surface at a second point a distance from the first point. By pulling the leading portion toward the trailing portion and securing the leading portion to the trailing portion proximate to the surface, expansion is limited in at least one portion of gastric tissue.

In embodiments, the primary elongate element is selected from the group consisting of: bands, wires, strings, threads, springs, ribbons, filaments, tubes, cables, yarn, strands, fibers, and ropes.

In embodiments, the primary elongate element has a cross-sectional shape selected from the group consisting of: round, ellipsoid, triangular, rectangular, and polygonal.

In embodiments, the primary elongate element possesses properties selected from the group consisting of: smooth, rough, serrated, barbed, beaded, spiked, undulate, elastic, and inelastic.

In embodiments, at least a portion of the first gastric portion is selected from the group consisting of a: fundus; body; antrum; and pylorus.

In embodiments, the limiting expansion in at least one portion of gastric tissue occurs in at least one of prior to gastric expansion, and during gastric expansion.

In embodiments, the method additionally includes, looping the primary elongate element around a gastric tissue second portion having a surface such that a third portion of the primary element exits the surface at a third point, the leading portion of the primary element exits the surface at a fourth point and pulling the leading portion toward the trailing portion proximate to the second portion gastric tissue surface.

In embodiments, at least a portion of the second gastric portion is selected from the group consisting of a: fundus; body; antrum; and pylorus.

In embodiments, the limiting expansion in at least one portion of gastric tissue occurs in at least one of: prior to gastric expansion and during gastric expansion.

In embodiments, the method additionally includes looping the primary elongate element around at least one third gastric tissue portion having a surface such that a fifth portion of the element exits the one third gastric tissue portion surface at a fifth point and the leading portion of the element exits the one third gastric tissue portion surface at a sixth point.

In embodiments, the at least one first, second, and third gastric tissue portions comprise an internal gastric surface and the securing is proximate to the internal surface.

In embodiments, the method additionally includes endoscopically performing at least one of the looping, the pulling, and the securing to the primary element described above.

In embodiments, the method additionally includes providing on at least one occasion, at least one of, unsecuring, pulling, and resecuring to at least one of the leading and trailing portions.

In embodiments, the at least one first, second, and third gastric tissue portions comprise an external gastric surface and the securing is proximate to the external surface.

In embodiments, the method additionally includes endoscopically performing at least one of the looping, the pulling, and the securing to the primary element described above.

In embodiments, the method additionally includes performing on at least one occasion, at least one of unsecuring, pulling, and resecuring to at least one of the leading and trailing portions, for example to adjust the extent of gastric expansion limitation as necessary.

In embodiments, the distance between at least one of the first and second points, third and fourth points, and fifth and sixth points, prior to the securing is at least about 2 centimeters, at least about 4 centimeters, at least about 6 centimeters, or even at least about 8 centimeters.

In embodiments, the distance between at least one of the first and second points, third and fourth points, and fifth and sixth points, following the securing is no more than about 20 centimeters, no more than about 15 centimeters, no more than about 10 centimeters, or even no more than about 8 centimeters.

In embodiments, the method additionally includes securing the loop in at least one of the first second and third gastric portions, thereby limiting expansion with the loop.

In embodiments, at least a portion of the at least one third gastric portion comprises a portion selected from the group consisting of a: fundus; body; antrum; and pylorus.

In embodiments, the limiting expansion in at least one portion of gastric tissue occurs in at least one of prior to gastric expansion, and during gastric expansion.

In embodiments, the method further comprises passing at least one ancillary elongate element having leading and trailing portions between at least two portions of the elongate element and two portions of gastric tissue selected from the group consisting of first second and third gastric portions, pulling the leading portion toward the trailing portion of the at least one ancillary element portion, and securing the leading portion to the trailing portion proximate to the tissue, thereby further limiting expansion in the gastric tissue.

In embodiments, the method further comprises, securing m loops of the elongate primary element in m respective portions of gastric tissue, m is at least four, looping n loops of the ancillary element between n+1 of the m loops of the primary element and respective gastric tissue portions, wherein “n” is at least three, thereby limiting expansion in the gastric tissue.

In embodiments, the method additionally includes endoscopically performing at least one of passing, pulling, and securing to the ancillary element described above.

In embodiments, the method additionally includes providing on at least one occasion, at least one of unsecuring, pulling, and resecuring to at least one of the ancillary leading and trailing portions, for example to adjust the extent of gastric expansion limitation as necessary.

In embodiments, at least one of the m loops is operatively associated with at least one gastric portion selected from the group consisting of a fundus, a body, an antrum and a pylorus.

In embodiments, the method additionally includes removeably securing each of the n loops of ancillary wire around m primary element portions.

In embodiments, the method additionally includes substantially permanently securing each of the n loops of ancillary wire around m primary element portions.

In embodiments, the limiting expansion in at least one portion of gastric tissue occurs in at least one of: prior to gastric expansion, and during gastric expansion.

In embodiments, the at least one ancillary elongate element is selected from the group consisting of: bands, wires, strings, threads, springs, ribbons, filaments, tubes, cables, yarn, strands, fibers, and ropes.

In embodiments, the at least one ancillary elongate element has a cross-sectional shape selected from the group consisting of round, ellipsoid, triangular, rectangular, and polygonal.

In embodiments, the at least one ancillary elongate element possesses properties selected from the group consisting of: smooth, rough, serrated, barbed, beaded, spiked, undulate, elastic, and inelastic.

In embodiments, the n loops of the ancillary element delineate a shape selected from the group consisting of: star, triangular, donut, ellipsoid, square and polygon.

In embodiments, the at least one ancillary elongate element comprises an inflatable component such as a balloon.

According to the teachings of the present invention, there is provided a gastric anchor comprising a body having first and second ends, a radially expandable head functionally associated with the first end of the body, the head configured to engage at least one portion of an internal gastric surface, an external gastric surface; and a tissue between the internal and external gastric surfaces, and an anchor base adapted to secure to the second end of the body.

In embodiments, the anchor is adapted to be deployed endoscopically.

In embodiments, the anchor the radial expansion is substantially parallel to the gastric surface.

In embodiments, the anchoring head consists essentially of at least one wire adapted to outwardly spiral from an origin at the body, the spiraling wire being radially compressible.

In embodiments, the anchoring head consists essentially of at least two wires adapted to extend radially outward from an origin at said body.

In embodiments, the shape of the spiral wire and the at least two radially extending wires are members of the group consisting of: round, ellipsoid, triangular, rectangular, and polygonal.

In embodiments, the primary elongate element has a cross-sectional shape selected from the group consisting of: round, ellipsoid, triangular, rectangular, and polygonal.

In embodiments the head comprises an expandable balloon, and the body includes a channel having an inlet port, in fluid communication with the expandable balloon.

In embodiments, the expandable balloon contains a polymerizable material. In embodiments, the polymerizable material configured to polymerize upon contact with a liquid. In embodiments, the polymer expands in volume upon contact with the liquid. In embodiments, the polymer comprises methyl methacrylate.

According to the teachings of the present invention, there is provided a method for limiting expansion in at least one portion of gastric tissue, the method comprising providing n gastric anchors, n is at least 2, each of the n gastric anchors the method additionally includes an anchor head, securing each of the n anchor heads, in a respective portion of gastric tissue, connecting each of the n anchors to at least one other of the n anchors using at least n−1 connectors, thereby limiting expansion of at least one gastric portion between any two connected anchors. In embodiments, n is at least three or even n is at least four.

In embodiments, n is at least four and at least one of the anchors is not coplanar with the at least three other anchors.

In embodiments, the method additionally includes endoscopically furnishing at least one of the providing, the securing; and the connecting to the at least one anchor.

In embodiments, the method additionally includes at least one of disconnecting; and reconnecting, at least one connector on at least one occasion.

In embodiments, the method additionally includes endoscopically performing at least one of disconnecting and/or reconnecting, at least one connector on at least one occasion, for example to adjust the extent of gastric expansion limitation as necessary.

In embodiments, the n anchors are connected with at least n connectors.

In embodiments, the primary elongate element has a cross-sectional shape selected from the group consisting of: round, ellipsoid, triangular, rectangular, and polygonal.

In embodiments, the n anchors are connected with at least n+1 connectors.

In embodiments, n−1 connectors are connected to a single anchor.

In embodiments, the distance between any two of the anchors prior to the securing is at least about 2 centimeters, at least about 4 centimeters, at least about 6 centimeters, or even at least about 8 centimeters.

In embodiments, the distance between any two of the anchors following the securing is no more than about 20 centimeters, no more than about 15 centimeters, no more than about 10 centimeters, or even no more than about 8 centimeters.

In embodiments, the limiting expansion in at least one portion of gastric tissue occurs in at least one of prior to gastric expansion, and during gastric expansion.

In embodiments the at least one gastric portion is selected from the group consisting of a fundus a body, an antrum, and a pylorus.

In embodiments, the limiting expansion in at least one portion of gastric tissue causes stimulation of at least one gastric satiety receptor.

In embodiments, the limiting expansion in at least one portion of gastric tissue prevents stimulation of at least one gastric hunger receptor.

In embodiments, at least of the n−1 anchors are positioned on at least one of an internal gastric surface, and an external gastric surface.

In embodiments, the at least n connectors are selected from the group consisting of: bands, wires, strings, threads, springs, ribbons, filaments, tubes, cables, yarn, strands, fibers, and ropes.

In embodiments, at least one of the connectors has a cross-sectional shape selected from the group consisting of: round, ellipsoid, triangular, rectangular, and polygonal.

In embodiments, at least one first and at least one second of the at least n connectors comprise a same type, and a different type from the group of types.

In embodiments, at least one of the at least n connectors is selected from the group of properties consisting of: smooth, rough, serrated, barbed, beaded, spiked, undulate, elastic, and inelastic.

In embodiments, at least one first and at least one second of the at least n connectors comprise a same property, and a different property, from the group of properties.

In embodiments, at least one of the at least n connectors comprises two connector elements, a first connector element and a second connector element, each of the two connector elements having leading and trailing portions, the trailing portions being connected to anchors and the leading portions being joined together.

In embodiments, at least a portion of at least one of the n anchors comprise a material selected from a group consisting of nitinol, stainless steel, shape memory materials, metals, polymers, polytetrafluoroethylene, polycarbonate, temperature curable materials, light-curable materials, water curable materials, and multi-component curable materials.

In embodiments, at least one first and at least one second of the n anchors comprise a same material, or a different material from the group of materials.

In embodiments, an anchoring portion of at least one of the n anchors comprises a mass of substantially fluid material and the securing further includes curing the fluid to form a cohesive mass.

In embodiments, the curing includes curing of at least one material taken from the group consisting of: adhesives, elastomers, and polymers, the polymers the method additionally includes light-curable polymers, polytetrafluoroethylene and polycarbonate.

According to the teachings of the present invention, there is provided a method for limiting expansion in at least one portion of gastric tissue, comprises securing n anchors, having base, body and head portions, to at least n gastric portions, n is at least two, connecting an anchor portions of n connectors to the n anchors, pulling terminal portions of the n connectors toward a locus located a distance from a gastric tissue surface, and securing the n terminal portions to each other at the locus, thereby limiting expansion in at least one of the n gastric portions.

In embodiments, the method additionally includes endoscopically securing at least one of the anchors.

In embodiments, the method additionally includes endoscopically providing to at least one of the connecting, the pulling; and the securing of at least one of the n connectors.

In embodiments, the method additionally includes, on at least one occasion, at least one of unsecuring, pulling and resecuring to at least one of the n connectors, for example endoscopically, for example to adjust the extent of gastric expansion limitation as necessary.

In embodiments, at least one of the n connectors is selected from the group consisting of: bands, wires, strings, threads, springs, ribbons, filaments, tubes, cables, yarn, strands, fibers, and ropes.

In embodiments, at least one of the n connectors has a cross-sectional shape selected from the group consisting of: round, ellipsoid, triangular, rectangular, and polygonal.

In embodiments, at least one of the n connectors possesses properties selected from the group consisting of: smooth, rough, serrated, barbed, beaded, spiked, undulate, elastic, and inelastic.

In embodiments, at least a portion of at least one of the n connectors includes properties selected from the group consisting of: smooth, rough, serrated, barbed, beaded, spiked, undulating, inflatable, elastic, and inelastic.

In embodiments, the method additionally includes pulling at least one first anchor toward at least one second anchor, placing at least one interconnector along an internal gastric surface, connecting the at least one first anchor to the at least one second anchor with at least one interconnector.

In embodiments, the method additionally includes endoscopically providing on at least one occasion the pulling, the placing, and the connecting to the at least one interconnector described above

In embodiments, the method additionally includes providing on at least one occasion disconnecting, pulling, and reconnecting to the at least one interconnector, for example endoscopically, for example to adjust the extent of gastric expansion limitation as necessary.

In embodiments, the at least one interconnector is selected from the group consisting of: bands, wires, strings, threads, springs, ribbons, filaments, tubes, cables, yarn, strands, fibers, and ropes.

In embodiments, the at least one interconnector has a cross-sectional shape selected from the group consisting of: round, ellipsoid, triangular, rectangular, and polygonal.

In embodiments, the at least one interconnector possesses properties selected from the group consisting of: smooth, rough, serrated, barbed, beaded, spiked, undulate, elastic, and inelastic.

In embodiments, at least a portion of the at least one interconnector includes properties selected from the group consisting of: smooth, rough, serrated, barbed, beaded, spiked, undulating, inflatable, elastic, and inelastic.

In embodiments, the connecting comprises directly connecting the terminal portions of the n connectors to each other.

In embodiments, the connecting comprises connecting the terminal portions of the n connectors to a connector element.

In embodiments, the connector element comprises a cone.

In embodiments, the cone includes at least one channel adapted to allow passage of fluid.

In embodiments, the cone includes at least one inflatable portion.

In embodiments, the method additionally includes endoscopically inflating at least one portion of the inflatable portion of the cone with a first inflation pressure.

In embodiments, the method additionally includes endoscopically inflating the at least one inflated portion to a second inflation pressure.

In embodiments, a cross section of the connector element comprises a shape selected from the group consisting of: round, ellipsoid, triangular, rectangular, and polygonal.

In embodiments, the connector element is selected from the group consisting of: smooth, rough, serrated, barbed, beaded, spiked, undulate, elastic, and inelastic.

In embodiments, the connector element includes properties selected from the group consisting of: smooth, rough, serrated, barbed, beaded, spiked, undulating, inflatable, elastic, and inelastic.

In embodiments, at least one head portion of the n anchors comprises a shape selected from the group consisting of an umbrella shape; a spiral shape, a ring shape, a plane, and a lens shape.

In embodiments, at least a portion of at least one of the n anchors comprises a material selected from a group consisting of nitinol, stainless steel, shape memory materials, metals, polymers, polytetrafluoroethylene, polycarbonate, temperature curable materials, light-curable materials, water curable materials, and multi-component curable materials.

In embodiments, at least one of the base, body and head portions of at least one of the n anchors is operatively associated with at least one of an internal gastric tissue surface, an external gastric tissue surface, an intermediate portion of gastric tissue.

In embodiments, at least one portion of the n gastric portions comprises a portion selected from the group consisting of a fundus; a body; an antrum; and a pylorus.

In embodiments, the limiting expansion occurs in at least one of: prior to gastric expansion and during gastric expansion.

According to the teachings of the present invention, there is provided a method for limiting expansion in at least one portion of gastric tissue, comprising placing at least one revolution of a substantially elongate resilient element around a gastric chamber, arranging the element into a substantially helical configuration, applying pressure with the element at four or more non-coplanar contact areas, the contact areas located on at least one of external gastric tissue, and internal gastric tissue.

In embodiments, at least a portion of the element applies pressure during at least one of prior to gastric expansion, and during gastric expansion.

In embodiments, the applying pressure with the element at the contact area occurs in at least one gastric portion selected from the group consisting of a fundus, a body, an antrum, and a pylorus.

In embodiments, the placing and arranging includes cooling the element and introducing the element into a catheter passage, exiting the element from a catheter passage, warming the element, and substantially coiling the element.

In embodiments, a cross section of at least a portion of the element is at least one of: substantially tubular: and substantially planar.

In embodiments, at least a portion of the element possesses properties selected from the group consisting of: smooth, rough, serrated, barbed, beaded, spiked, undulate, elastic and inelastic.

In embodiments, the element includes at least two protrusions along its length.

In embodiments, the two or more protrusions comprise a shape selected from the group consisting of a sphere, a pyramid and a fiat surface.

In embodiments, the method additionally includes adjusting the pressure applied to at least two of the contact areas during gastric expansion.

In embodiments, the method additionally includes adjusting the pressure applied to at least two of the contact areas prior to gastric expansion.

In embodiments, the method additionally includes interposing an interposition between the element and at least a portion of the contact area and a gastric surface.

In embodiments, the interposition comprises at least one of an organic interposition, and a non-organic interposition.

There is thus provided a method for constricting at least one portion of gastric tissue, comprises folding at least one portion of gastric tissue to form a substantially elongate ridge, bending an elongate piece of material into a clamping trough shape having an apex and two walls extending from the apex, and clamping the ridge between the two walls.

In embodiments, the method additionally includes endoscopically providing at least one of the folding, the bending and the clamping to the ridge.

In embodiments, the clamping trough comprises a material selected from a group consisting of nitinol, stainless steel, shape memory materials, metals, polymers, polytetrafluoroethylene, polycarbonate, temperature curable materials, light-curable materials, water curable materials and multi-component curable materials.

In embodiments, at least a portion of the clamping trough possesses properties selected from the group consisting of: smooth, rough, serrated, barbed, beaded, spiked, undulate, elastic and inelastic.

In embodiments, the ridge is formed along at least one portion of at least one of: an internal gastric tissue surface and an external gastric surface.

In embodiments, at least a portion of the ridge is oriented at an angle relative to the longitudinal gastric axis of at least one of between about 0 degrees and about 20 degrees, between about 20 degrees and about 70 degrees, and between about 70 degrees and about 90 degrees.

In embodiments, the method further comprises passing a securing component through at least one of the two walls and the tissue clamped there between.

In embodiments, the securing component is selected from the group consisting of: pins, staples, rods, pegs, suture, thread, bands, wires, strings, threads, springs, ribbons, filaments, tubes, cables, yarn, strands, fibers, and ropes.

In embodiments, the securing component has a cross-sectional shape selected from the group consisting of: round, ellipsoid, triangular, rectangular, and polygonal.

In embodiments, the apex includes at least two junctions at least one first junction between the apex and the first wall, and at least one second junction between the apex and the second wall, the second junction located a distance from the first junction.

In embodiments, the distance between the first and the second junctions is at least one of flat, and curved.

In embodiments, at least a portion of the apex is curved.

In embodiments, the apex of the clamping trough is at least one of straight and spiral.

According to the teachings of the present invention, there is provided a method for bypassing a gastric chamber, comprising providing a substantially tubular gastric bypass sleeve, the sleeve defining a lumen and having leading and trailing portions, securing the leading portion so that at least a portion of the lumen is substantially continuous with an esophageal lumen, connecting the trailing portion so that at least a portion of the lumen is substantially continuous with an pyloric lumen, and opening at least one opening through the sleeve between the esophageal and the pyloric lumens, the at least one opening providing communication between at least a portion of a first material in the lumen and at least a portion of a second material in the gastric chamber.

In embodiments, the method additionally includes endoscopically performing at least one of the providing, the securing, the connecting, and the opening to the gastric bypass sleeve described above.

In embodiments, the method further comprises extending the leading portion beyond an esophageal sphincter, while ensuring substantial esophageal sphincter function.

In embodiments, the method further comprises extending the trailing portion beyond a pyloric sphincter, while ensuring substantial pyloric sphincter function.

In embodiments, the securing at least one of the leading and trailing portions comprises applying an outwardly radial force on the sleeve from within the lumen, for example with the use of an expandable stent.

In embodiments, the securing at least one of the leading and trailing portions comprises placing an outwardly expandable substantially tubular element within the lumen and expanding the element.

In embodiments, the expanding element is chosen from a material selected from a group consisting of nitinol, stainless steel, shape memory materials, metals, polymers, polytetrafluoroethylene, polycarbonate, temperature curable materials, light-curable materials, water curable materials, and multi-component curable materials.

In embodiments, the method further comprises placing at least one adjustable constrictor around a portion of the bypass sleeve, and applying a constricting force of a first magnitude around the portion.

In embodiments, the method additionally includes endoscopically furnishing at least one of the placing and the applying to the at least one bypass sleeve.

In embodiments, the method additionally includes adjusting the constricting force to a second magnitude.

In embodiments, the method additionally includes endoscopically adjusting the constricting force to a second magnitude.

In embodiments, the at least one adjustable constrictor includes a material selected from a group consisting of nitinol, stainless steel, shape memory materials, metals, polymers, polytetrafluoroethylene, polycarbonate, temperature curable materials, light-curable materials, water curable materials, and multi-component curable materials.

In embodiments, the at least one adjustable constrictor possesses properties selected from the group consisting of: smooth, rough, serrated, barbed, beaded, spiked, undulate, inflatable, elastic, and inelastic.

In embodiments, the at least one adjustable constrictor comprises a shape selected from the group consisting of: round, ellipsoid, triangular, rectangular, and polygonal.

According to the teachings of the present invention, there is provided a gastric expansion restrictor adapted to be endoscopically deployed through a gastric catheter, the restrictor comprises at least four elongate elements, each element comprises an elongate body having a first end extending from an origin common to all of the at least four elongate elements, a second end from which a tissue connector extends such that when assembled, the at least four tissue connectors are adapted to connect to at least four portions of gastric tissue and when the at least four tissue connectors are connected to the at least four portions of gastric tissue, at least one of the connectors is adapted to assume a non coplanar position with respect to at least three of the tissue connectors.

In embodiments, the origin includes a shaped body that has a shape from the group consisting of star, round, ellipsoid, triangular, rectangular, and polygonal.

In embodiments, the shaped body has a cross sectional shape from the group consisting of star, round, ellipsoid, triangular, rectangular, and polygonal.

In embodiments, the elongate body portion is selected from the group consisting of: bands, wires, strings, threads, springs, ribbons, filaments, tubes, cables, yarn, strands, fibers, and ropes.

In embodiments, the elongate body portion has a cross-sectional shape selected from the group consisting of round, ellipsoid, triangular, rectangular, and polygonal.

In embodiments, the elongate body portion possesses properties selected from the group consisting of: smooth, rough, beaded, undulate, elastic, and inelastic.

In embodiments, each of the elongate body portions has a length of at least about 2 centimeters, at least about 4 centimeters, at least about 6 centimeters, or even at least about 8 centimeters.

In embodiments, each of the elongate body portions has a length of no more than about 20 centimeters no more than about 20 centimeters, no more than about 15 centimeters, no more than about 10 centimeters, or even no more than about 8 centimeters.

In embodiments, the tissue connector possesses properties selected from the group consisting of: smooth, rough, beaded, undulate, elastic, and inelastic.

In embodiments, the tissue connector is selected from the group consisting of: hooks, loops, barbs, and clips.

### BRIEF DESCRIPTION OF THE DRAWINGS

The present invention providing methods and devices for limiting gastric expansion so as to affect motility, volume, hunger sensation and/or nutrient absorption is described by way of example with reference to the accompanying drawings. With specific reference now to the drawings in detail, it is stressed that the particulars shown are by way of example and for purposes of illustrative discussion of the preferred method of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the methods of the invention may be embodied in practice.

FIGS. 1 and 2 depict a suture deployed in a stomach configured to provide gastric expansion limitation (and in embodiments concomitant satiety receptor stimulation) in a cross section of the stomach, in accordance with an embodiment of the present invention;

FIGS. 3, 4, 6, and 7 show embodiments of gastric anchors deployed in a stomach in accordance with embodiment of the present invention;

FIGS. 5, 8 and 9 show the gastric anchors of FIGS. 3, 4, 7 and 8 configured to provide gastric expansion limitation in a cross section of the stomach, in accordance with an embodiment of the present invention;

FIG. 10 shows a gastric funnel deployed in a stomach configured to provide gastric expansion limitation in a cross section of the stomach, in accordance with an embodiment of the present invention;

FIGS. 11 and 12 show an endoscopically deployable device for gastric expansion limitation in a cross section of the stomach, in accordance with an embodiment of the present invention;

FIG. 13 shows a helical band deployed external to the stomach and configured to provide gastric expansion limitation, in accordance with an embodiment of the present invention;

FIGS. 14, 15, and 16 show gastric clamping troughs applied to internal stomach tissue shown in cross section in accordance with an embodiment of the present invention;

FIG. 17 shows multiple gastric clamping troughs applied to external stomach tissue in accordance with an embodiment of the present invention; and

FIG. 18 shows a gastric bypass sleeve in a cross section of a stomach in accordance with an embodiment of the present invention.

### DESCRIPTION OF THE PREFERRED EMBODIMENTS

In broad terms, the present invention relates to methods and devices for limiting gastric expansion so as to affect gastric motility, gastric volume, satiety sensation, hunger sensation and/or nutrient absorption

The principles and uses of the teachings of the present invention may be better understood with reference to the accompanying description, figures and examples. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of biology, engineering, material sciences, medicine and physics. Such techniques are thoroughly explained in the literature.

Unless otherwise defined, all technical and scientific terms used herein have the same meaning as commonly understood by one of ordinary skill in the art to which the invention belongs. In addition, the descriptions, materials, methods, and examples are illustrative only and not intended to be limiting. Methods and materials similar or equivalent to those described herein can be used in the practice or testing of the present invention.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, “a” or “an” mean “at least one” or “one or more”. The use of the phrase “one or more” herein does not alter this intended meaning of “a” or “an”.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Implementation of the methods of the present invention involves performing or completing selected tasks or steps manually, automatically, or a combination thereof.

### Gastric Suture

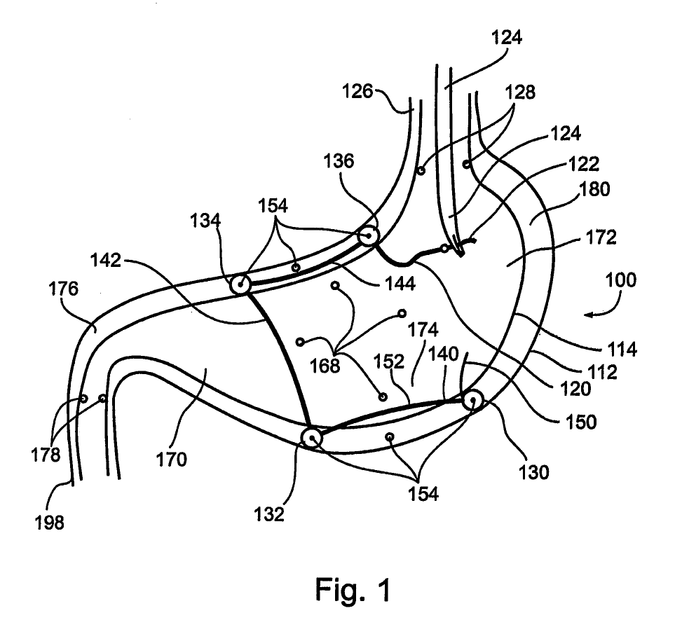

FIG. 1 shows a stomach 100 in which a suture 120 is secured in a gastric tissue portion 180 shown in cross section. Suture 120 is optionally passed through tissue 180 using a curved suture needle 122 that is held by a forceps 124. Forceps 124 is typically deployed during minimally invasive endoscopic procedures, often in conjunction with a gastroscope, using techniques that are well known to those familiar with the art.

Suture 120 is passed through tissue 180 using a short loop 130 whose ends, 150 and 152, are optionally secured to each other under tension. In the configuration shown, loop 130 compresses tissue 180, so that pressure is applied to a gastric hunger receptor 154, thereby preventing receptor 154 from registering a lack of food within stomach 100 that would cause a sensation of hunger.

Similarly, small loops 132, 134 and 136 are looped in a manner that provides pressure on receptors 154. Optionally, at least one of loops 130, 132, 134 and 136 is non coplanar with the remaining three of loops 130, 132, 134 and 136.

Long suture lengths 140, 142 and 144, span between loops 130, 132, 134 and 136, thereby limiting expansion of tissue 180 adjacent to each of lengths 140, 142 and 144. As a result of limitation of expansion, the volume of stomach 100 that expands upon food intake is reduced so that stomach expansion receptors 168 register reaching a maximum volume with a minimum of food intake, thereby providing the subject with a feeling of fullness.

In embodiments, where lengths 140, 142 and 144 are left relatively loose, limitation of expansion occurs only after the onset of expansion of stomach 100 at which time the subject will feel fullness due to stimulation of receptors 168 and/or 154.

In other embodiments where lengths 140, 142 and 144 are tightened to restrict stomach tissue 180 even when stomach 100 is at a minimal volume, stomach 100 will register fullness even with intake of a small volume of food that stimulate receptors 168 and/or 154.

The present invention contemplates application of the above to a variety of gastric-related tissue 180. For example, suture 120 is passed through a variety of tissues of gastric-related tissue 180, including an esophagus 126, an esophageal sphincter 128, a fundus 172, an antrum 170, a body 174, a pylorus 176 and/or a pyloric sphincter 178, particularly in areas of high density of receptors 154 and 168.

The exact mechanisms of providing satiety and fullness sensations to an obese individual are not fully known to the bariatric community. It is believed that restricting volume of stomach 100 causes receptors 154 and 168 to register satiation and/or fullness, thereby favorably influencing diet and aiding in weight loss.

Any reference to receptors 154 and 168 a priori refers to any gastric receptors presently identified and those that will be identified, for example by bariatric researchers, in the future. Additionally, the methods and/or configuration of material applied to stomach 100, for example size and/or placement of loops 130, 132, 134 and 136 and/or suture lengths 140, 142 and 144, a priori include any modifications that are discovered to be efficacious or become known in the future.

Generally, systems that limit expansion of stomach 100—in at least one portion of gastric tissue 180 where expansion limiting occurs during either or both prior to gastric expansion resulting from eating and during gastric expansion resulting from eating—result in weight loss only for a given period, for example two years. Habituation, wherein receptors 154 and 168 fail to register signals of satiation or fullness respectively, is a possible cause for obese individuals stopping losing weight.

To reduce habituation, tension along lengths 140, 142 and 144 and/or loops 130, 132, 134 and 136 is increased or decreased endoscopically at a later date following initial deployment.

As used herein gastric tissue 180 refers to any portion of gastric-related tissue 180 that is part of, or near, stomach 100, for example, inter alia, esophagus 126, fundus 172, antrum 170, body 174, pylorus 176, pyloric sphincter 178 and/or an intestine 198.

As used herein gastric tissue 180 adjacent to a material implanted in gastric tissue 180, for example loops 130, 132, 134 and 136, and/or suture lengths 140, 142 and 144, refers to tissue 180 directly below the material and portions of tissue extending there from that are influenced by suture 120, for example in limiting expansion.

As used herein limiting gastric expansion refers to applying materials, for example suture lengths 140, 142 and 144 that limit volume of stomach 100 either at rest or as stomach 100 fills with food. Limiting expansion of gastric tissue 180 additionally refers to any effects that occur as a result of expansion limitation, including, inter alia, limiting gastric motility, hunger sensation and/or nutrient absorption. The benefits of the configuration presented, using lengths 140, 142 and 144 and loops 130, 132, 134 and 136 are not restricted to weight loss. For example, by placing suture 120 near esophageal sphincter 128, suture 120 may be configured to treat gastroesophogeal reflux disease (GERD).

### Suture Specifications

The present invention contemplates a variety of materials and configurations for gastric suture 120.

In embodiments, suture 120, alternatively referred to as a primary elongate element, is an element such as one or more bands, wires, strings, threads, springs, ribbons, filaments, tubes, cables, yarn, strands, fibers, and ropes.

In embodiments, suture 120 has any suitable cross-sectional shape such as round, ellipsoid, triangular, rectangular, or polygonal.

In embodiments, suture 120 possesses properties such as smooth, rough, serrated, barbed, beaded, spiked, undulate, elastic, and/or inelastic.

In embodiments, lengths 140, 142 and 144 are dispensed on the outside of stomach 100, herein referred to as proximate to an external surface 112, rather than internal surface 114.

In embodiments, and as noted above, sometime after loops 130, 132, 134, and 136 are formed and secured, for example anywhere from a few days or more, a second procedure is performed in which loops 130, 132, 134, and 136 are unsecured, tension is adjusted and loops 130, 132, 134, and 136 are re-secured. Optionally, the second procedure is performed endoscopically.

In embodiments, the distance between loops 130 and 132; between loops 132 and 134; between loops 134 and 136 prior to tightening lengths 140, 142 and 144 is at least about 2 centimeters, at least about 4 centimeters, at least about 6 centimeters, or even at least about 8 centimeters.

In embodiments, the distance between loops 130 and 132; between loops 132 and 134; between loops 134 and 136 following tightening lengths 140, 142 and 144 is at least about 2 centimeters, at least about 4 centimeters, at least about 6 centimeters, or even at least about 8 centimeters.

In embodiments, the distance between loops 130 and 132; between loops 132 and 134; between loops 134 and 136 prior to tightening lengths 140, 142 and 144 is no more than about 20 centimeters, no more than about 15 centimeters, no more than about 10 centimeters, or even no more than about 8 centimeters.

### Ancillary Expansion Limiters

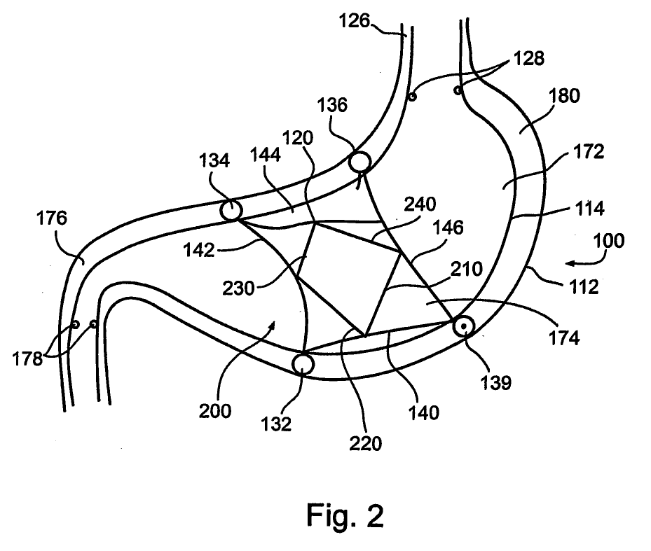

FIG. 2 shows suture 120 used in conjunction with an ancillary expansion limiter 200 comprising a first suture span 210 that provides tension on length 140 and on a length 146. First suture span 210 is alternatively referred to as ancillary elongate element 210. Similarly, a second span 220, a third span 230 and a fourth span 240 connect from lengths 140 to 142, 142 to 144, and 144 to 146 respectively.

Spans 210, 220, 230, and 240 are typically tightened to create greater gastric expansion limitation than with lengths 140, 142, 144 and 146 alone.

Spans 210, 220, 230, and 240 additionally supply another tightening option at a future endoscopic procedure by which gastric tissue 110 and/or volume of stomach 100 is further adjusted, for example by limiting or expanding gastric expansion.

While in FIG. 2 only four spans 210, 220, 230, and 240 that are connected end to end in four lengths 140, 142, 144 and 146 that are connected end to end are depicted, it is understood that the number and configuration of lengths 140, 142, 144 and 146 and spans 210, 220, 230, and 240 is not limited and is determined by the health care professional who is implanting the spans.

In embodiments, at least one span is non-coplanar to at least three other spans. For example, in FIG. 2 the four spans 210, 220, 230, and 240 are not coplanar and rather substantially define a tetrahedron.

In embodiments, spans 210, 220, 230, and 240 limit expansion in at least one portion of gastric tissue 180 where expansion limiting occurs during either or both prior to gastric expansion resulting from eating and during gastric expansion resulting from eating.

### Ancillary Expansion Limiters Specifications

In embodiments, spans 210, 220, 230, and 240 are such elements as one or more of bands, wires, strings, threads, springs, ribbons, filaments, tubes, cables, yarn, strands, fibers, and ropes.

Spans 210, 220, 230, and 240 have any suitable cross-sectional shape such as round, ellipsoid, triangular, rectangular, or polygonal.

In embodiments, spans 210, 220, 230, and 240 possess properties such as smooth, rough, serrated, barbed, beaded, spiked, undulate, elastic, and/or inelastic.

In embodiments, spans 210, 220, 230, and 240 together delineate a shape such as a star, triangular, donut, ellipsoid, square or polygon.

### Radially Expanding Gastric Anchors

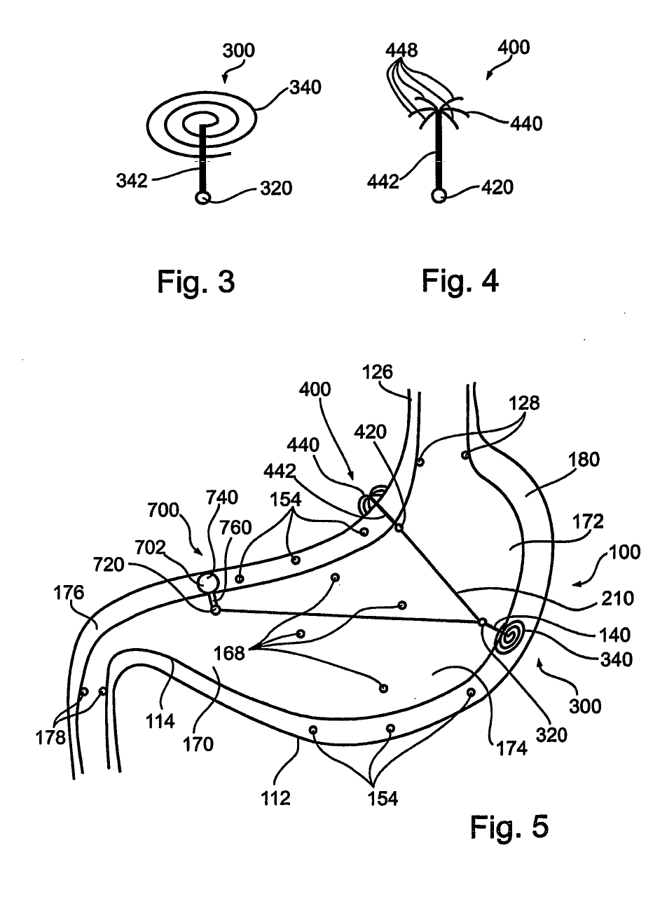

FIGS. 3, 4, 6 and 7 depict embodiments of gastric anchors of the present invention 300, 400 and 700. FIGS. 5 and 8 depict methods of implantation of gastric anchors 300, 400, and 700, in accordance with an embodiment of the present invention.

Gastric anchor 300 (FIG. 3) comprises a spiraling wire (substantially a logarithmic spiral) head 340 extending from an anchor body 342 including an anchor ring 320. In embodiments, spiraling wire head 340 is radially compressible and the radial expansion of spiraling wire head 340 is substantially parallel to gastric tissue portion 180 (FIG. 5)

Gastric anchor 400 (FIG. 4) comprises an anchoring head 440 having multiple wires 448 extending radially outward from an anchor body 442. Anchoring head 440 is radially compressible. Anchor body 442 further includes an anchor ring 420.

As seen in FIG. 5, gastric anchors 300 and 400 have been deployed in gastric tissue 180 and connected with suture length 140 using, for example, an endoscopic technique.

Anchor heads 340 and 440 are adapted to substantially form a plane that is substantially parallel to gastric tissue portion 180 when expanded. While gastric anchor 300 is shown deployed within tissue 180, in embodiments anchor 300 is deployed external to tissue 180. Similarly, while gastric anchor 400 is shown deployed external to tissue 180, in embodiments anchor 400 is deployed within tissue

### Radially Expanding Gastric Anchors Specifications

In embodiments, a wire 340 is formed into a spiral. Suitable spirals include logarithmic, round, ellipsoid, triangular, rectangular, and polygonal spirals.

In embodiments, anchor bodies 342 and 442 have cross-sectional shapes selected from the group consisting of round, ellipsoid, triangular, rectangular, and polygonal. In embodiments, anchors 300 and 400 comprise heads 340 and 440 having alternative shapes, for example an umbrella shape, a spiral shape, a ring shape, a plane, or a lens shape.

In embodiments, at least a portion of anchor 300 and/or 400 comprise a material such as nitinol, stainless steel, shape memory materials, metals, polymers, polytetrafluoroethylene, polycarbonate, temperature curable materials, light-curable materials, water curable materials, and/or multi-component curable materials.

### Balloon Gastric Anchors

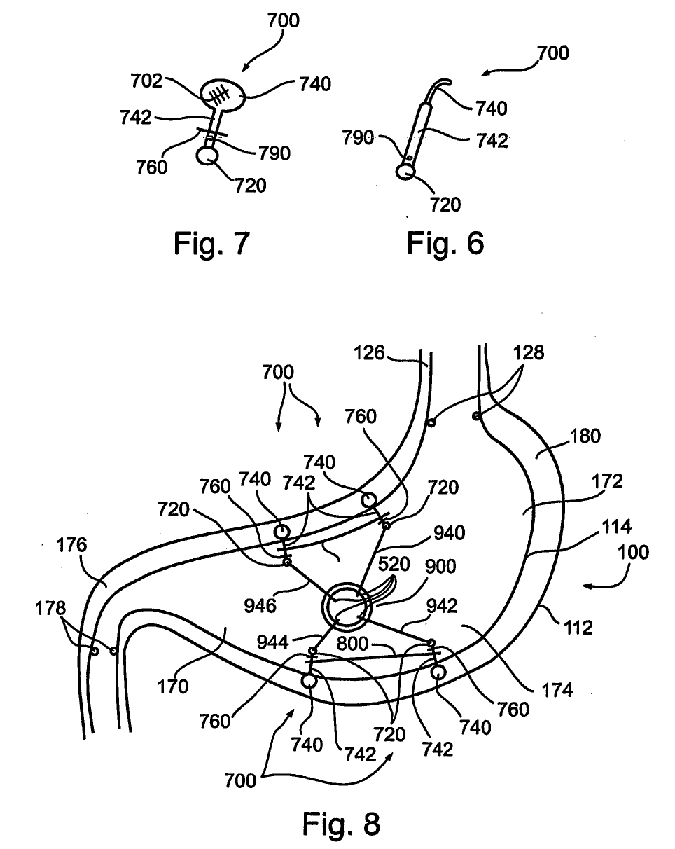

FIGS. 6, 7, 8, and 9 show embodiments and methods of implantation of a gastric anchor including an expandable anchoring head, gastric anchor 700 provided with balloon anchor head 740, in accordance with an embodiment of the present invention. Anchor 700 comprises a body 742 having a ring 720 at a first end and an expandable balloon 740 at a second end. In embodiments, body 742 includes an inlet port 790 in fluid communication with expandable balloon 740.

In FIG. 6 balloon anchor head 740 is in a deflated configuration and in FIG. 7 in an inflated configuration. While head 740 is shown as a singular sac extending from body 742, head 740 may alternatively have any number of configurations. For example, head 740 may include 2, 3 or even 6 multiple tubular rays that extend radially from body 742 and expand against or within a gastric tissue portion.

Alternatively, head 740 may have a round, ellipsoid, triangular, rectangular, or polygonal cross-sectional shape.

Additionally, head 740 may comprise a material without rubber and/or rubber-like qualities, for example a non-stretchable plastic sheet material formed into a hollow inflatable chamber.

In embodiments, expandable balloon 740 contains a polymerizable material 702. In embodiments, polymerizable material 702 is configured to polymerize upon contact with a liquid. In embodiments, polymerizable material 702 expands in volume upon contact with the liquid. In embodiments, polymerizable material 702 comprises methyl methacrylate.

### Balloon Gastric Anchor Deployment

In FIG. 8, anchor 700 has been deployed in stomach 100 by expanding balloon 740 within gastric tissue 180 and using a cross piece 760 to anchor against interconnectors 800.

Additionally, wire lengths 940, 942, 944 and 946 have been secured to rings 720 at a first end and to a central ring 900 at a second end. In embodiments, ring 900 includes receptacles 820 to which wire lengths 940, 942, 944 and 946 can be adjustably secured.

In embodiments, gastric tissue 180 under interconnectors 800 is compressed between anchors 700, while wire lengths 940, 942, 944 and 946 pull interconnectors 800 to further limit expansion of stomach 100 in at least one portion of gastric tissue 180 where expansion limiting occurs during either or both prior to gastric expansion resulting from eating and during gastric expansion resulting from eating.

In embodiments, following a period after initial installation, wire lengths 940, 942, 944 and 946 are tightened or loosened, preferably endoscopically, to reduce or expand volume of stomach 100.

In embodiments, rings 320, 420, and/or 720 (FIGS. 3, 4, and 7) are connected to one or more first ends of wire lengths 940, 942, 944 and 946. Second ends of wire lengths 940, 942, 944 and 946 are either joined together or secured to a central body, for example adjustable connector 820.

Balloon Gastric Anchors with Interconnectors

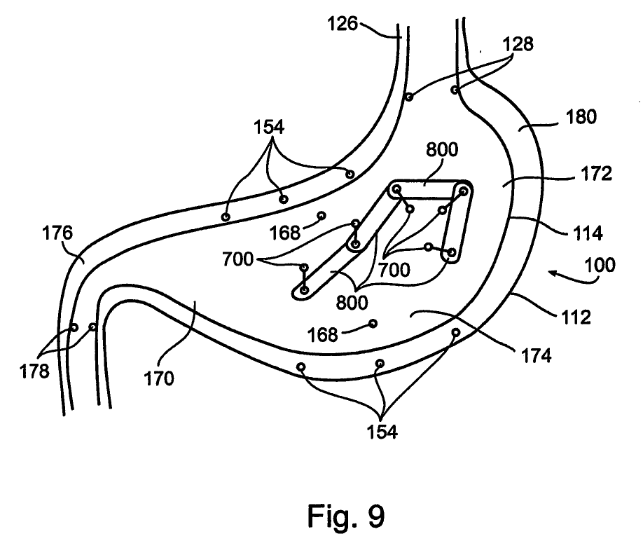

In FIG. 9 anchors 700 have been deployed in stomach 100 to anchor interconnectors 800 against gastric tissue 180. As in FIG. 8, gastric tissue 180 under interconnectors 800 is compressed between anchors 700.

With respect to anchors 300, 400 and 700, in embodiments, at least n anchors 300, 400 and 700 are deployed; where n is at least four and at least one of anchors 300, 400 and/or 700 is not coplanar with the at least three other anchors 300, 400 and/or 700.

### Balloon Interconnector Specifications

Interconnector 800 is any suitable element such as one or more of bands, wires, strings, threads, springs, ribbons, filaments, tubes, cables, yarn, strands, fibers, and ropes.

Interconnector 800 has any suitable cross-sectional shape such as round, ellipsoid, triangular, rectangular or polygonal

In embodiments, at least a portion of interconnector 800 includes properties such as smooth, rough, serrated, barbed, beaded, spiked, undulating, inflatable, elastic, and inelastic.

### Gastric Cone

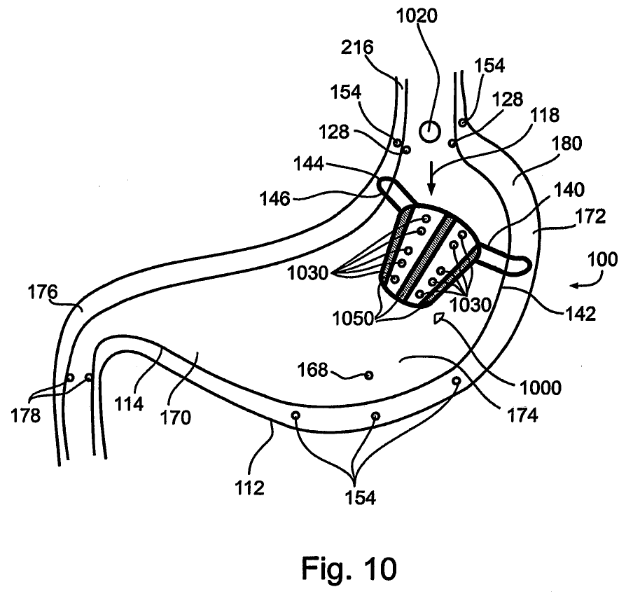

FIG. 10 shows an alternative configuration of ring 900 shown in FIG. 8, comprising a gastric cone 1000 configured to slow down the movement of a food bolus 1020 passing in a direction 118. During slowing, pressure from food bolus 1020 presses against satiation receptors 154 located near esophageal sphincter 128, providing a feeling of satiation.

In embodiments, cone 1000 includes openings 1030 that allow passage of fluids in direction 118 toward pyloric sphincter 178, thereby gradually allowing digested portions of food bolus 1020 to pass cone 1000.

Optionally openings 1030 form a mesh or net shaped, for example, into a band that encircles cone 1000. In embodiments, the band that includes mesh openings 1030 has a height of at least about one centimeter and, for example, no more than about 10 centimeters. In embodiments, the band of encircling mesh openings 1030 allows exchange of fluids between cone 1000 and gastric tissue 180. Mesh openings 1030, additionally may comprise a different material than other portions of cone 1000, for example comprising metallic and/or polymeric materials.

In embodiments, openings 1030 have an area of at least about 0.5 mm², 0.75 mm² or about 1.0 mm². In embodiments, openings 1030 have an area of no more than about 2.0 mm², about 1.0 mm², about 1.25 mm², or about 1.50 mm².

In embodiments, cone 1000 includes inflatable sections 1050 that are endoscopically inflatable and, depending on the degree of expansion, provide a greater or lesser degree of slowing movement of bolus 1020.

### Multi-Strand Gastric Restrictor

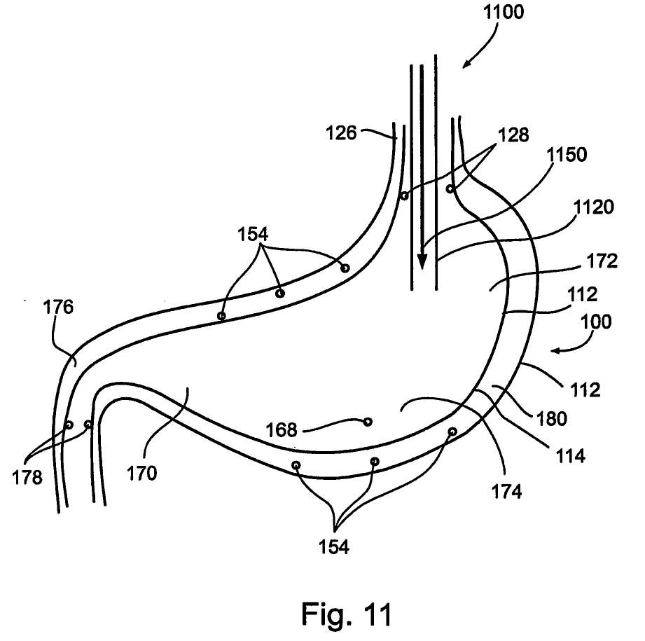

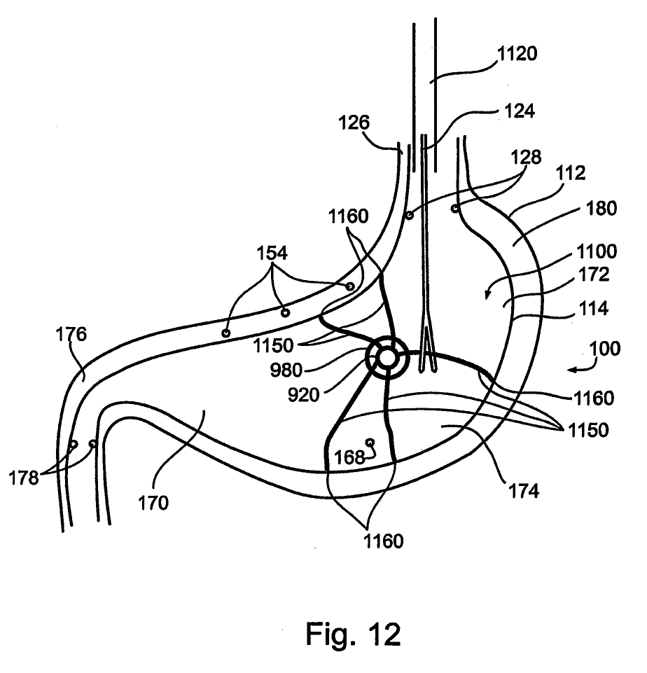

FIGS. 11 and 12 show a Multi-Strand Gastric Restrictor 1100 during deployment in a cross section of stomach 100 from a gastric tube 1120. Multi-Strand Gastric Restrictor 1100 comprises central ring 900 from which spokes 1150 extend. Spokes 1150 additionally have hooks 1160 that are secured in gastric tissue 180 using endoscopically deployed forceps 124.

In embodiments, each spoke 1150 includes multiple hooks 1160 extending, for example, radially outward form a central origin at spoke 1150. Multiple hooks 1060 extending from spoke 1150 provide multiple attachment points to gastric tissue 180 thereby spreading the load on each portion of gastric tissue 180 to which hooks 1150 are attached. Spreading the load helps to prevent pullout of hooks 1150 from a respective portion of gastric tissue 180.

In other embodiments, each spoke 1150 includes multiple strands (not shown) that, for example, extend radially outward from spoke 1150, each strand ending in at least one hook 1160 that attaches to gastric tissue 160.

In embodiments, spokes 1150, alternatively referred to as elongate elements 1150, are adjustable and connected to central ring 900.

### Multi-Strand Gastric Restrictor Specifications

In embodiments, ring 900, also referred to as origin 900, includes a shaped body 920 that has any suitable shape such as star, round, ellipsoid, triangular, rectangular, or polygonal shaped. Spokes 1150, alternatively referred to as elongate body portions 1150, are any suitable element, such as one or more of bands, wires, strings, threads, springs, ribbons, filaments, tubes, cables, yarn, strands, fibers, and ropes.

Spokes 1150 have any suitable cross-sectional shape such as round, ellipsoid, triangular, rectangular or polygonal.

In embodiments, spokes 1150 possess properties such as smooth, rough, beaded, undulate, elastic, and/or inelastic.

In embodiments, spokes 1150 have a length of at least about 2 centimeters, at least about 4 centimeters, at least about 6 centimeters, or even at least about 8 centimeters.

In embodiments, spokes 1150 have a length of no more than about 20 centimeters, no more than about 20 centimeters, no more than about 15 centimeters, no more than about 10 centimeters, or even no more than about 8 centimeters.

In embodiments, there are at least four spokes 1150.

In embodiments, hooks 1160, alternatively referred to as tissue connectors 1160, possess properties such as smooth, rough, beaded, undulate, elastic, and inelastic.

In embodiments, hooks 1160 are components such as hooks, loops, barbs, eyelets and clips.

In embodiments, hooks 1160 are connected to the at least four portions of gastric tissue 180, at least one of hooks 1160 is adapted to assume a non coplanar position with respect to at least three of the tissue connectors.

### Gastric Helical Banding

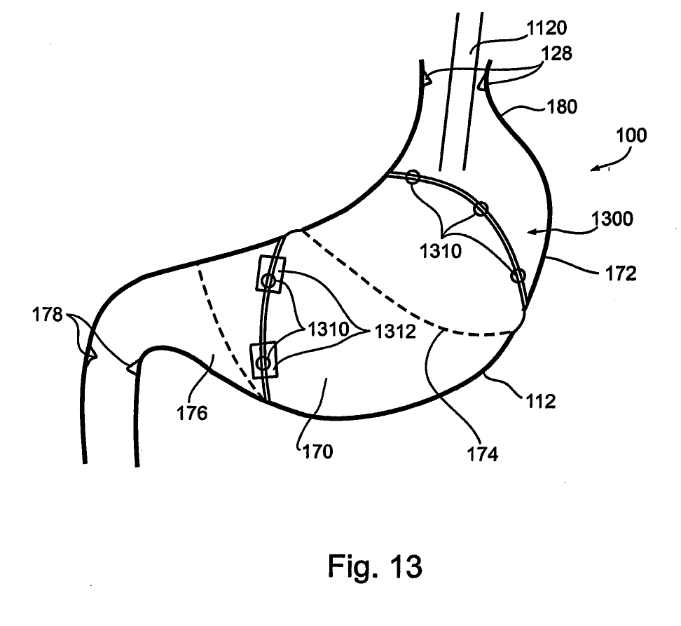

FIG. 13 shows a helical band 1300 deployed external to stomach 100 and configured to limit expansion of stomach 100, in accordance with an embodiment of the present invention.

In embodiments, helical band 1300 comprises at least one revolution of a substantially elongate resilient element around stomach 100 and applies pressure to four or more non-coplanar areas along areas including esophageal sphincter 128, fundus 172, antrum 170, body 174, pylorus 176 and/or pyloric sphincter 178. In an alternative embodiment, band 1300 is located internal to stomach 100 (not shown).

In embodiments, at least a portion of helical band 1300 applies pressure prior to gastric expansion and/or during gastric expansion.

In embodiments, band 1300 comprises a shape memory material that, upon heating, regains a shape. In such embodiments, deployment of band 1300 includes cooling band 1300 and introduction through a gastric tube passage 1120, exiting band 1300 from passage 1120, warming band 1300 to substantially attain a helical coiled shape adapted to contact stomach 100 as noted above.

### Gastric Helical Band Specifications

In embodiments, a cross section of helical band 1300 is at least one of substantially tubular, and substantially planar.

In embodiments, helical band 1300 possesses properties such as smooth, rough, serrated, barbed, beaded, spiked, undulate, elastic, and inelastic.

In embodiments, helical band 1300 includes at least two protrusions 1310 along its length.

In embodiments, protrusions 1310 comprise a shape such as a sphere, a pyramid, and/or a flat surface.

In embodiments, deployment additionally includes adjusting the pressure applied by at least two of protrusions 1310 during gastric expansion.

In embodiments, deployment additionally includes adjusting the pressure applied by at least two of protrusions 1310 prior to gastric expansion.

In embodiments, deployment additionally includes interposing at least one interposition 1312 between helical band 1300 and at least a portion of stomach 100. Interpositions 1312 serve, for example, to increase limitation in expansion of gastric tissue 180 and/or provide a cushion between protrusions 1310 and gastric tissue 180.

In embodiments, interposition 1312 comprises at least one of an organic interposition, and a non-organic interposition.

### Gastric Clamping Troughs

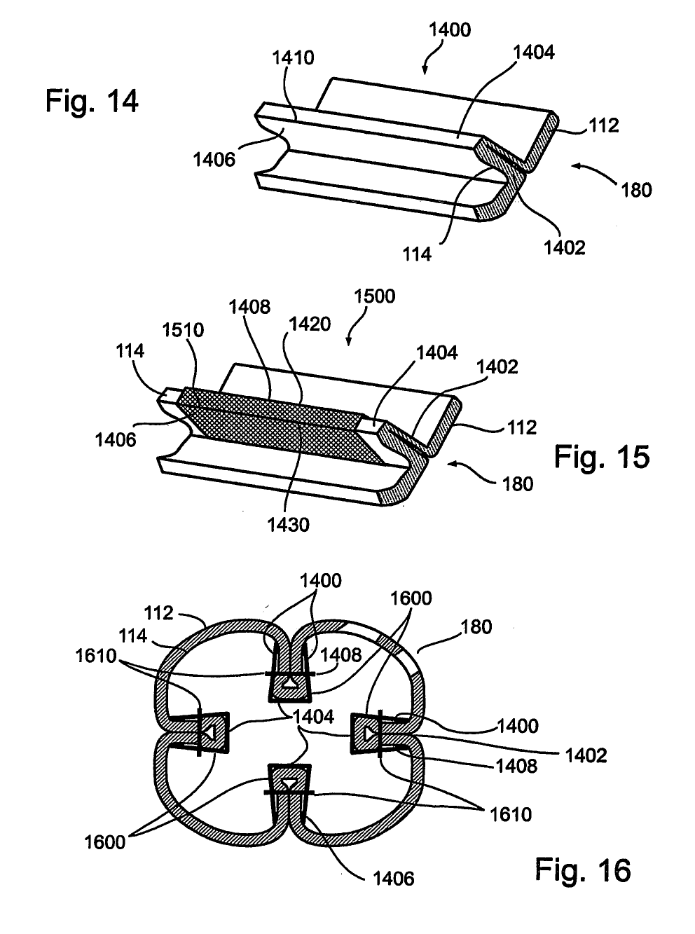

FIGS. 14, 15, and 16 show gastric clamping troughs 1400, 1500 and 1600 applied to internal stomach tissue 180, shown in cross section, in accordance with an embodiment of the present invention.

Gastric clamping trough 1400 comprises a solid material 1410 while clamping trough 1500 comprises a mesh 1510. FIG. 16 shows clamping troughs 1600 each having a cross pin 1610 that passes through gastric tissue 180. Cross pin 1610 aids in maintaining clamping troughs 1600 firmly placed around tissue 180.

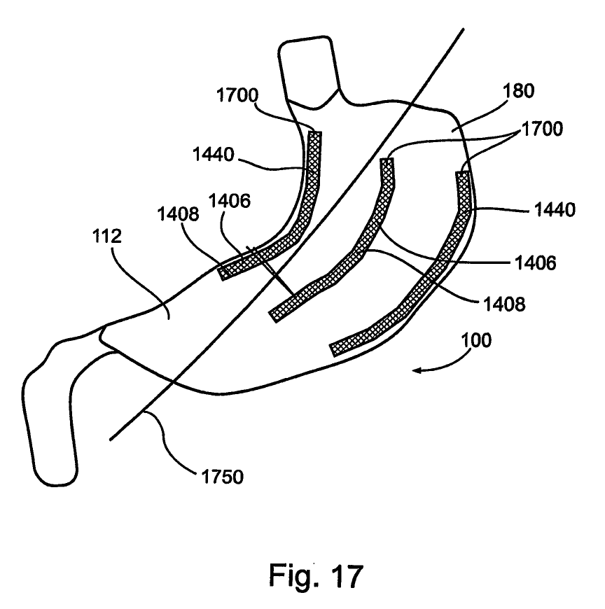

FIG. 17 shows multiple gastric clamping troughs 1700 applied to external tissue portion 180 of stomach 100.

In further embodiments, clamping trough 1700 forms a spiral, for example around stomach, thereby limiting expansion of gastric tissue 180.

A method for providing clamping troughs 1400, 1500, 1600 and 1700 includes folding at least one portion of gastric tissue 180 to form a substantially elongate ridge 1402 (FIGS. 14, 15, and 16), bending an elongate piece of material into a clamping trough shape 1400, 1500, 1600 and 1700. Clamping trough shapes 1400, 1500, 1600 and 1700 include an apex 1404 and two walls 1406 and 1408 extending from apex 1404, and clamping 1402 ridge between walls 1406 and 1408.

In embodiments, the method additionally includes endoscopically performing at least one of folding, bending, and clamping to ridge 1402 during said providing.

In further embodiments, clamping troughs 1400, 1500, 1600 and/or 1700 are compressed against ridge 1402 in a manner that prevents blood from flowing through ridge 1402. In such embodiments, ridge 1402 will optionally anastomose, fusing as a permanent ridge 1402. Additionally, due to the reduced blood supply resulting from the increased pressure, ridge 1402 will optionally atrophy.

In embodiments, with ridge 1402 substantially atrophied, clamping troughs 1400, 1500, 1600 and/or 1700 loosen and fall off ridge 1402 and pass through the gut to be excreted. In such cases, ridge 1402 will remain fused, thereby maintaining limitation of expansion of gastric tissue 180 without additional devices.

### Gastric Clamping Trough Specifications

In embodiments, clamping troughs 1400, 1500, 1600 and 1700 comprise a material such as nitinol, stainless steel, shape memory materials, metals, polymers, polytetrafluoroethylene, polycarbonate, temperature curable materials, light-curable materials, water curable materials, and multi-component curable materials.

In embodiments, clamping troughs 1400, 1500, 1600 and 1700 possess properties such as smooth, rough, serrated, barbed, beaded, spiked, undulate, elastic, and inelastic.

In embodiments, ridges 1402 are formed along internal surface of gastric portion 180. Alternatively, ridges 1402 are formed along external surface of gastric portion 180.

In embodiments, at least a portion of ridge 1402 is oriented at an angle relative to a longitudinal gastric axis 1750 (FIG. 17) of at least one of between about 0 degrees and about 20 degrees, between about 20 degrees and about 70 degrees, and between about 70 degrees and about 90 degrees.

In embodiments, the method further comprises passing a crosspiece 1610, alternatively referred to as a securing component 1610 through at least one of two walls 1406 and 1408 and tissue 180 clamped there between.

Additionally, clamping troughs 1400, 1500, 1600 and/or 1700 may be endoscopically adjusted in the amount of pressure applied to ridge 1402 at implantation and/or at a later date.

In embodiments, ridge 1600, securing component 1610 may be installed having a first length, thereby providing a first amount of pressure on tissue 180. At a future date, for the purpose of changing the pressure on ridge 1402, securing component 1610 of a different length is endoscopically installed.

In embodiments, securing components 1610 in a variety of lengths are additionally provided in a kit along with ridge 1600 so that the installer has the option to apply a specific amount of pressure to ridge 1402 based upon the length of component 1610 chosen.

In embodiments, securing component 1610 is any suitable element such as one or more of is selected from the group consisting of pins, staples, rods, pegs, sutures, bands, wires, strings, threads, springs, ribbons, filaments, tubes, cables, yarn, strands, fibers, and ropes.

Securing component 1610 has any suitable cross-sectional shape such as round, ellipsoid, triangular, rectangular, and polygonal. In embodiments, apex 1404 (FIG. 15) includes a first junction 1420 between apex 1404 and first wall 1408 and a second junction 1430 between apex 1404 and second wall 1406.

In embodiments, the distance between first 1420 and second 1430 junctions is at least one of flat, and curved.

In embodiments, at least a portion of apex 1404 is curved.

In embodiments, apex 1404 is at least one of straight and spiral.

### Gastric Bypass Sleeve

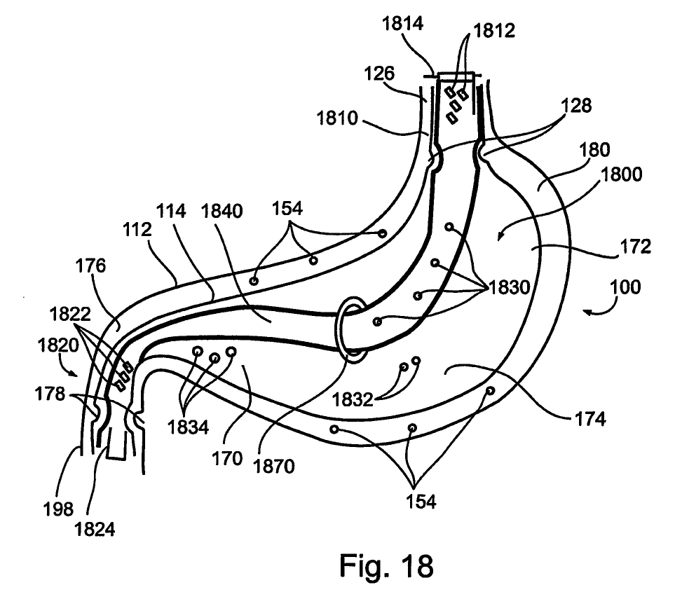

FIG. 18 shows a gastric bypass sleeve 1800 in a cross section of stomach 100, in accordance with an embodiment of the present invention. Bypass sleeve includes an esophageal portion 1810 comprising a flexible material that conforms to the action of esophageal sphincter 128 and a pyloric portion 1820 comprising a flexible material that conforms to the action of pyloric sphincter 178.

For example, bypass 1800 optionally has

a) esophageal bendable sections 1812 at esophageal sphincter 128 that allow substantial closing of sphincter 128, thereby preventing reflux from stomach 100 to esophagus 126; and

b) pyloric bendable sections at pyloric sphincter 178 that allow substantial closing of sphincter 178, thereby preventing reflux from intestinal 198 to stomach 100.

Additionally, bypass sleeve 1800 includes one or more openings 1830 designed to allow exchange of digestive material 1832, including digestive enzymes from gastric tissue 180 and/or nutrients 1834 from within the contents of gastric bypass sleeve 1800.

In embodiments, sleeve 1800 includes at least one restrictor 1870, for example being configured to adjustably tighten around sleeve 1800, thereby selectively changing the passage speed of nutrients 1834 and/or enzymes 1832, through sleeve 1800.

According to the teachings of the present invention, there is provided a method for bypassing a gastric chamber 100, comprising providing a substantially tubular gastric bypass sleeve 1800, comprising a body 1840 and connecting esophageal portion 1810 of said body 1840 to esophagus 126 and pyloric portion 1820 to pylorus 176. In embodiments, the method includes providing at least one opening 1830 through body 1840, providing communication between at least a portion of digestive material 1832 originating in gastric tissue 180 and nutrients 1834 originating within sleeve 1800.

In embodiments, the method additionally includes endoscopically providing sleeve 1800 and making the above-noted connections to esophagus 126 and pylorus 176.

In embodiments, the method further comprises extending esophageal sleeve portion 1810 beyond esophageal sphincter 128, while ensuring substantial function of esophageal sphincter 128.

In embodiments, the method further comprises extending the pyloric sleeve portion 1820 beyond a pyloric sphincter 178, while ensuring substantial function of pyloric sphincter 178.

In embodiments, following the above-noted extending, pyloric sleeve portion 1820 is secured to pylorus 176, optionally with an outwardly radially expanding element 1814 placed within a lumen of pyloric portion 1820.

In embodiments, esophageal sleeve portion 1810 is secured to esophagus 126, optionally with outwardly radially expanding esophageal element 1814 placed within lumen of esophageal portion 1810.

In embodiments, expanding elements 1814 and 1824 are of a suitable material such as nitinol, stainless steel, shape memory materials, metals, polymers, polytetrafluoroethylene, polycarbonate, temperature curable materials, light-curable materials, water curable materials, and multi-component curable materials.

In embodiments, the method further comprises placing at least one adjustable constrictor 1870 around a portion of bypass sleeve 1800, and applying a constricting force of a first magnitude around sleeve 1800.

In embodiments, the method additionally includes endoscopically furnishing and/or constricting sleeve 1800.

In embodiments, the method additionally includes adjusting constrictor 1870 to apply constricting force of a second magnitude. Optionally, the adjusting is made endoscopically.

### Gastric Bypass Sleeve Specifications

In embodiments, adjustable constrictor 1870 includes a suitable material such as nitinol, stainless steel, shape memory materials, metals, polymers, polytetrafluoroethylene, polycarbonate, temperature curable materials, light-curable materials, water curable materials, and multi-component curable materials.

In embodiments, adjustable constrictor 1870 possesses properties such as smooth, rough, serrated, barbed, beaded, spiked, undulate, inflatable, elastic, and inelastic.

In embodiments, adjustable constrictor 1870 comprises a shape such as round, ellipsoid, triangular, rectangular, and polygonal. It is expected that during the life of this patent many relevant delivery systems will be developed and the scope of the various embodiments of gastric restrictor 1100, anchors 300, 400 and 700, clamping troughs 1400, 1500, 1600 and 1700, and cone 1000 and the various methods of implementation are intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad to scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for limiting expansion in at least one portion of gastric tissue, comprising:
a) looping an elongate primary element around a substantially continuous first gastric tissue portion having a surface such that:
a leading portion of said primary element exits a first gastric tissue surface at a first point;
a trailing portion of said primary element exits a second gastric tissue surface at a second point a distance from said first point;
b) pulling said leading portion toward said trailing portion; and
c) securing said leading portion to said trailing portion proximate to said surface,
thereby limiting expansion in at least one portion of gastric tissue.

2. (canceled)

3. The method according to claim 1, wherein said limiting expansion in at least one portion of gastric tissue, occurs in at least one of:
i) prior to gastric expansion; and
ii) during gastric expansion.

4. The method according to claim 1, additionally including:
d) prior to step “c”, looping said primary elongate element around a gastric tissue second portion having a surface such that:
a third portion of said primary element exits said surface at a third point;
said leading portion of said primary element exits said surface at a fourth point; and
e) pulling said leading portion toward said trailing portion proximate to said second portion gastric tissue surface.

5-6. (canceled)

7. The method according to claim 4, additionally including:
f) prior to step “c”, looping said primary elongate element around at least one third gastric tissue portion having a surface such that:
a fifth portion of said element exits said one third gastric tissue portion surface at a fifth point; and said leading portion of said element exits said one third gastric tissue portion surface at a sixth point.

8. The method according to claim 7, wherein said at least one:
first;
second; and
third gastric tissue portions comprise an internal gastric surface and said securing is proximate to said internal surface.

9. The method according to claim 8, including:
h) performing on at least one occasion, at least one of;
unsecuring;
pulling; and
resecuring to at least one of said leading and trailing portions.

10-11. (canceled)

12. The method according to claim 7, including securing said loop in at least one of said first second and third gastric portions, thereby limiting expansion with said loop.

13. (canceled)

14. The method according to claim 7, wherein said limiting expansion in at least one portion of gastric tissue, occurs in at least one of:
i) prior to gastric expansion; and
ii) during gastric expansion.

15. The method according to claim 7, further comprising:
g) passing at least one ancillary elongate element having leading and trailing portions between at least two portions of said elongate element and two portions of gastric tissue selected from the group consisting of first second and third gastric portions;
h) pulling said leading portion toward said trailing portion of said at least one ancillary element portion; and
i) securing said leading portion to said trailing portion proximate to said tissue, thereby further limiting expansion in said gastric tissue.

16. The method according to claim 15, further comprising, following step “i”:
j) securing m loops of said elongate primary element in m respective portions of gastric tissue, wherein m is at least four;
k) looping n loops of said ancillary element between n+1 of said m loops of said primary element and respective gastric tissue portions, wherein “n” is at least three,
thereby limiting expansion in said gastric tissue.

17-20. (canceled)

21. The method according to claim 16, wherein said limiting expansion in at least one portion of gastric tissue, occurs in at least one of:
i) prior to gastric expansion; and
ii) during gastric expansion.

22-46. (canceled)

47. A method for limiting expansion in at least one portion of gastric tissue, comprising:
a) securing n anchors, having base, body and head portions, to at least n gastric portions, wherein n is at least two;
b) connecting an anchor portions of n connectors to said n anchors;
c) pulling terminal portions of said n connectors toward a locus located a distance from a gastric tissue surface; and
d) securing said n terminal portions to each other at said locus, thereby limiting expansion in at least one of said n gastric portions.

48-49. (canceled)

50. The method according to claim 47, including:
d) pulling at least one first anchor toward at least one second anchor;
e) placing at least one interconnector along an internal gastric surface;
f) connecting said at least one first anchor to said at least one second anchor with at least one interconnector.

51. The method according to claim 47, wherein said connecting comprises directly connecting said terminal portions of said n connectors to each other.

52. The method according to claim 47, wherein said connecting comprises connecting said terminal portions of said n connectors to a connector element.

53. The method according to claim 52, wherein said connector element comprises a cone.

54. The method according to claim 52, wherein said cone includes at least one channel adapted to allow passage of fluid.

55-59. (canceled)

60. The method according to claim 47, wherein said limiting expansion occurs in at least one of:
i) prior to gastric expansion; and
ii) during gastric expansion.

61-65. (canceled)

66. A method for constricting at least one portion of gastric tissue, comprising:
a) folding at least one portion of gastric tissue to form a substantially elongate ridge;
b) bending an elongate piece of material into a clamping trough shape having an apex and two walls extending from said apex; and
c) clamping said ridge between said two walls.

67. (canceled)

68. The method according to claim 66, wherein said ridge is formed along at least one portion of at least one of:
i) an internal gastric tissue surface; and
ii) an external gastric surface.

69. (canceled)

70. The method according to claim 66, further comprising passing a securing component through at least one of said two walls and said tissue clamped there between.

71-74. (canceled)

75. The method according to claim 66, wherein said apex of said clamping trough is at least one of:
i) straight; and
ii) spiral.

76. A method for bypassing a gastric chamber, comprising:
a) providing a substantially tubular gastric bypass sleeve, said sleeve defining a lumen and having leading and trailing portions;
b) securing said leading portion so that at least a portion of said lumen is substantially continuous with an esophageal lumen;
c) connecting said trailing portion so that at least a portion of said lumen is substantially continuous with a pyloric lumen; and
d) opening at least one opening through said sleeve between said esophageal and said pyloric lumens, said at least one opening providing communication between at least a portion of a first material in said lumen and at least a portion of a second material in said gastric chamber.

77-80. (canceled)

81. The method according to claim 76, further comprising:
g) placing at least one adjustable constrictor around a portion of said bypass sleeve; and
h) applying a constricting force of a first magnitude around said portion.

82-84. (canceled)

85. A gastric expansion restrictor adapted to be endoscopically deployed through a gastric catheter, said restrictor comprising at least four elongate elements, each element comprising:
i) an elongate body having a first end extending from an origin common to all of said at least four elongate elements;
ii) a second end from which a tissue connector extends, such that when assembled, said at least four tissue connectors are:
a) adapted to connect to at least four portions of gastric tissue, and
b) when said at least four tissue connectors are connected to said at least four portions of gastric tissue, at least one of said connectors is adapted to assume a non coplanar position with respect to at least three of said tissue connectors.
